(19) United States
(12) Patent Application Publication
(10) Pub. No.: US 2026/0137397 A1
(43) Pub. Date: May 21, 2026

(54) BURR FOR SURGICAL PROCEDURES

(71) Applicant: Forma Medical, Inc., Camp Hill, PA (US)

(72) Inventors: Jesse F. Doty, Hixson, TN (US); James A. Gault, Lincoln, RI (US); Andrew Charles Davison, Hummelstown, PA (US)

(73) Assignee: Forma Medical, Inc., Camp Hill, PA (US)

(21) Appl. No.: 19/391,171

(22) Filed: Nov. 17, 2025

Related U.S. Application Data
(60) Provisional application No. 63/722,799, filed on Nov. 20, 2024.

(51) Int. Cl. A61B 17/16

(52) U.S. Cl. CPC A61B 17/1615

(57) ABSTRACT

An embodiment of the present disclosure is a surgical device. The surgical device includes a shaft elongated along a central axis. The shaft has a proximal end and a distal end spaced from the proximal end along the central axis. The surgical device includes a burr at the distal end of the shaft. The burr has a body defining a leading end, a trailing end, cutting flutes that extend from the leading end toward the trailing end and spiral around the central axis, and a cross-sectional dimension that his perpendicular to the central axis. The leading end of the burr has a greater cross-sectional dimension that any portion of the burr head that is proximal to the leading end. Another embodiment is a method for using the surgical device as described herein.

### CROSS-REFERENCE TO RELATED APPLICATIONS

The present application claims the benefit of and priority to U.S. Provisional Application No. 63/722,799, filed Nov. 20, 2024, the entire contents of which are incorporated by reference in this application.

### TECHNICAL FIELD

The present disclosure relates to a burr used for surgical procedures, such as surgical procedures for the extremity bones, such as the bones in the arms, lower legs and feet.

### BACKGROUND

Surgical burrs cut away tissue in preparation for implanting an anchor or other device in various orthopedic procedures. Typical surgical burrs taper toward its leading end so that the leading end has a smaller diameter than the portion of the burr proximal to the leading end. But such a shape is not suitable for certain procedures in the foot and hand.

### SUMMARY

There is a need for surgical burr with a burr that tapers toward the central axis in proximal direction. An embodiment of the present disclosure surgical device. The surgical device includes a shaft elongated along a central axis. The shaft has a distal end and a proximal end spaced from the distal end along the central axis in a proximal direction. The device also includes a burr at the distal end of the shaft. The burr has aa body defining a leading end, a trailing end, cutting flutes that extend from the leading end toward the trailing end and spiral around the central axis. The burr has a cross-sectional dimension that his perpendicular to the central axis. The leading end of the burr has a greater cross-sectional dimension than any portion of the burr that is proximal to the leading end.

In the embodiment above, the surgical device may include a groove that extends into the body of the burr and twists along an outer surface thereof.

In the embodiment above, the shaft has a first cross-sectional dimension, the leading end of the burr has a second cross-sectional dimension that is greater than the first cross-sectional dimension, and the trailing end of the burr has a third cross-sectional dimension that is less than the second cross-sectional dimension of the burr. The second cross-sectional dimension of the leading end may be between 1.5 mm and 4 mm.

In the embodiment above, the leading end of the burr has a distal-most surface, where the distal-most surface is blunt. And the distal-most surface is substantially perpendicular to the central axis.

In the embodiment above, the leading end of burr has a distal-most surface, where the distal-most surface defines a semi-spherical shape.

In the embodiment above, the leading end of burr has a distal-most surface, where the distal-most surface defines a conical shape.

In the embodiment above, the leading end of burr has a distal-most surface, where the distal-most surface defines a frustoconical shape.

An embodiment of the present disclosure surgical device that includes a power driver with a motor and a coupling. The device also includes a surgical burr having a shaft elongated along a central axis. The shaft has a distal end and a proximal end spaced from the distal end along the central axis in a proximal direction. The distal end is engageable with the coupling of the power driver. The surgical device has a burr at the distal end of the shaft. The burr has a body defining a leading end, a trailing end, cutting flutes that extend from the leading end toward the trailing end and spiral around the central axis, and a cross-sectional dimension that his perpendicular to the central axis. The leading end of the burr has a greater cross-sectional dimension than any portion of the burr that is proximal to the leading end.

In the embodiment above, the surgical device may include a groove that extends into the body of the burr and twists along an outer surface thereof.

In the embodiment above, the shaft has a first cross-sectional dimension, the leading end of the burr has a second cross-sectional dimension that is greater than the first cross-sectional dimension, and the trailing end of the burr has a third cross-sectional dimension that is less than the second cross-sectional dimension of the burr. A cross-sectional dimension of the leading end is between 1.5 mm and 4 mm.

In the embodiment above, the leading end of the burr has a distal-most surface, where the distal-most surface is blunt. The distal-most surface is substantially perpendicular to the central axis.

In the embodiment above, the leading end of burr has a distal-most surface, where the distal-most surface defines a semi-spherical shape.

In the embodiment above, the leading end of burr has a distal-most surface, where the distal-most surface defines a conical shape.

In the embodiment above, the leading end of burr has a distal-most surface, where the distal-most surface defines a frustoconical shape.

Another embodiment includes a method. The method also includes coupling a surgical burr to a driver, where the surgical burr, the surgical burr having a leading end, a trailing end, cutting flutes that extend from the leading end toward the trailing end and spiral around a central axis, and a cross-sectional dimension that his perpendicular to the central axis, where the leading end of the surgical burr has a greater cross-sectional dimension that any portion of the surgical burr that is proximal to the leading end. The method also includes positioning the burr into a target site of an extremity bone. The method also includes powering the driver to cause the burr to resect or cut the extremity bone.

In the embodiment above, the method may include distracting a joint of the target site before positioning the surgical burr into the target site.

In the embodiment above, the method may include a groove that extends into the body of the burr and twists along an outer surface thereof.

### BRIEF DESCRIPTION OF THE DRAWINGS

FIG. 1 a perspective view of a surgical burr according to an embodiment of the present disclosure;

FIG. 2 is a front view of the surgical burr shown in FIG. 1;

FIG. 3 is a back view of the surgical burr shown in FIG. 1;

FIG. 4 is a left view of the surgical burr shown in FIG. 1;

FIG. 5 is a right view of the surgical burr shown in FIG. 1;

FIG. 6 is a top view of the surgical burr shown in FIG. 1;

FIG. 7 is a bottom view of the surgical burr shown in FIG. 1;

FIG. 8 a perspective view of a surgical burr according to another embodiment of the present disclosure;

FIG. 9 is a front view of the surgical burr shown in FIG. 8;

FIG. 10 is a back view of the surgical burr shown in FIG. 8;

FIG. 11 is a left view of the surgical burr shown in FIG. 8;

FIG. 12 is a right view of the surgical burr shown in FIG. 8;

FIG. 13 is a top view of the surgical burr shown in FIG. 8;

FIG. 14 is a bottom view of the surgical burr shown in FIG. 8;

FIG. 15 a perspective view of a surgical burr according to another embodiment of the present disclosure;

FIG. 16 is a front view of the surgical burr shown in FIG. 15;

FIG. 17 is a back view of the surgical burr shown in FIG. 15;

FIG. 18 is a left view of the surgical burr shown in FIG. 15;

FIG. 19 is a right view of the surgical burr shown in FIG. 15;

FIG. 20 is a top view of the surgical burr shown in FIG. 15;

FIG. 21 is a bottom view of the surgical burr shown in FIG. 15;

FIG. 22 a perspective view of a surgical burr according to another embodiment of the present disclosure;

FIG. 23 is a front view of the surgical burr shown in FIG. 22;

FIG. 24 is a back view of the surgical burr shown in FIG. 22;

FIG. 25 is a left view of the surgical burr shown in FIG. 22;

FIG. 26 is a right view of the surgical burr shown in FIG. 22;

FIG. 27 is a top view of the surgical burr shown in FIG. 22; and

FIG. 28 is a bottom view of the surgical burr shown in FIG. 22.

### DETAILED DESCRIPTION OF ILLUSTRATIVE EMBODIMENTS

Embodiments of the present disclose include a surgical burr and related devices for performing surgical procedures on extremity bones, such as the foot and hands. The surgical burrs described herein may be used with power drivers used in surgery for the foot and/or hands. The power drivers as described herein may include a power unit including a motor, a burr handpiece (or coupler), and various cables that transmit power from the unit the burr handpiece. The burr handpiece receives in its distal end the proximal end of the shaft of the burrs as described herein. Once the burr is in place in the handpiece, the system can make cuts or remove tissue from the intended surgical site.

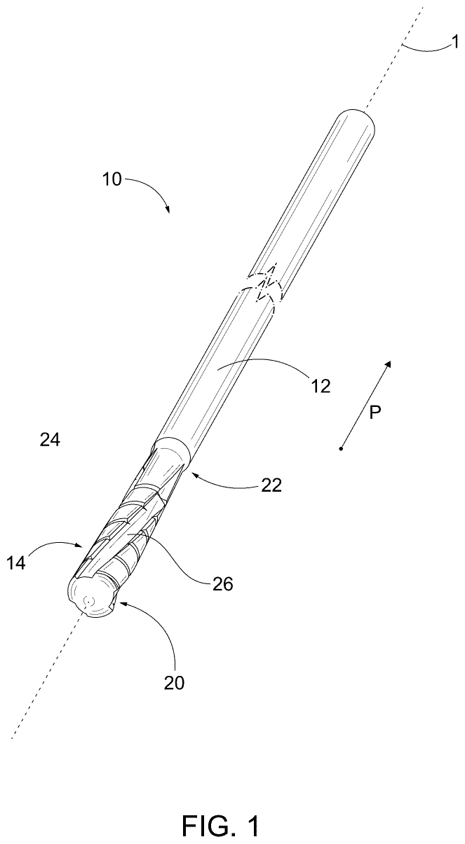

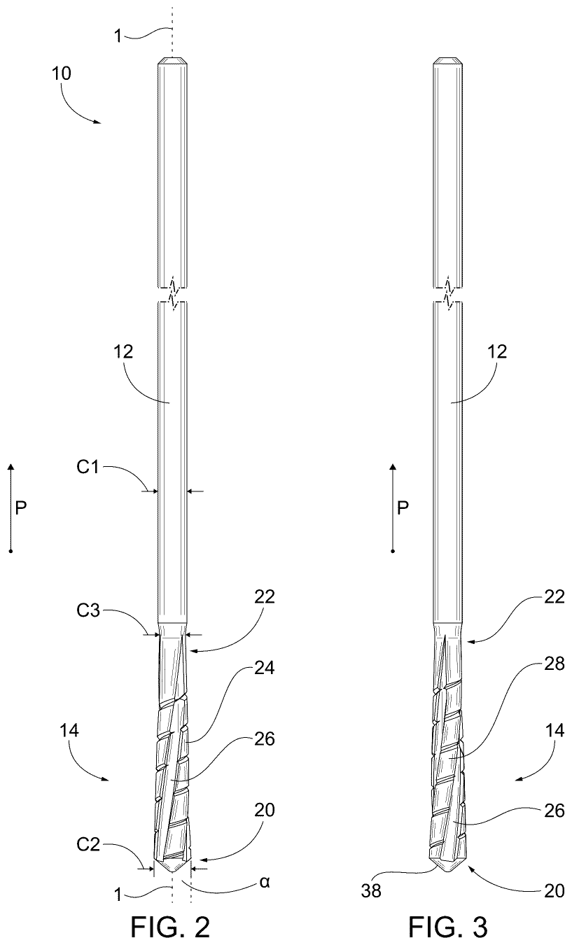

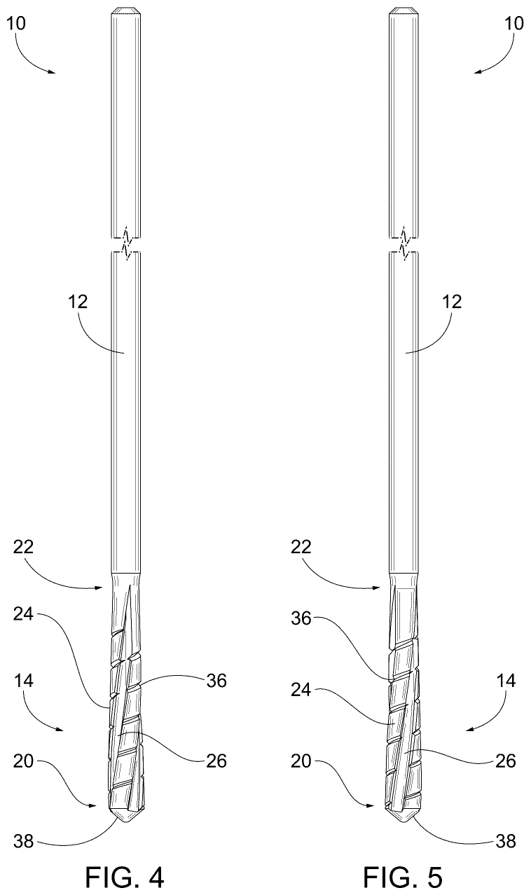

Referring to FIGS. 1-7, an embodiment of the surgical burr device 10 includes a shaft 12 elongated along a central axis. The shaft 12 has a proximal end 16 and a distal end 18 spaced from the proximal end 16 along the central axis 1. The device 10 also includes a burr 14 at the distal end of the shaft 12. The burr 14 has a body defining a leading end 20, a trailing end 22, and a cross-sectional dimension C that is perpendicular to the central axis 1. As shown, the leading end 20 of the burr 14 has a greater cross-sectional dimension than any portion of the burr that is proximal to the leading end. In other words, the burr 14 is tapered toward the central axis 1 as it extends toward the shaft 12 in a proximal direction P (or rearward relative to the leading end) along the central axis 1. This creates a burr shape that is a so-called reverse wedge, which is opposite of typical burrs that are tapered toward the leading end of the burr. In addition, the taper of the burr 14 has a taper angle a, which is the angle formed between the central axis 1 and the sloping outer surface of the leading end 20 of the burr 14. The taper angle in this instance can between about 3 to 5° and about 45°. The taper angle could vary from this range as needed.

In addition, the cross-sectional dimensions of the surgical device 10 vary along its length with the burr 14 being a reverse taper. More specifically, the shaft 12 has a first cross-sectional dimension C1 (which is perpendicular to the central axis 1), sometime referred as the shaft cross-sectional dimension C1. The leading end 20 of the burr 14 has a second cross-sectional dimension C2 (sometimes referred to as the leading end cross-sectional dimension) that is greater than the first cross-sectional dimension C1 of the shaft 12. The trailing end 22 of the burr has a third cross-sectional dimension C3 (sometimes referred to as the trailing end cross-sectional dimension) that is less than the second cross-sectional dimension C2. In addition, the third cross-sectional dimension C3 may be less than the first cross-sectional dimension C1 of the shaft. In one example, the leading end cross-sectional dimension C2 is between 1.5 mm and 4 mm. In another example, the leading end cross-sectional dimension C2 is 2 mm. In yet another example, the leading end cross-sectional dimension C2 is 2.5 mm. In yet another example, the leading end cross-sectional dimension C2 is 3.5 mm.

The benefit of burr, shaped as a reverse wedge or cone, as described herein is that for certain procedures such as a Lapidus, which is procedure to treat bunions affecting the big toe joint. In this case, where a closing wedge is made, the surgeon can insert the burr from the medial side, which is a clinical benefit and improved ease of use. Thus, more bone may be removed on the lateral side of the joint, which will allow the surgeon to reduce the distal metatarsal head laterally for an anatomic alignment. Currently, surgeons need to freehand this from the dorsal side with a burr. Thus, this new burr allows for greater procedure flexibility and improves clinical outcomes, for example, by reducing or minimizing variance associated with freehand removal of certain areas of tissue during these procedures.

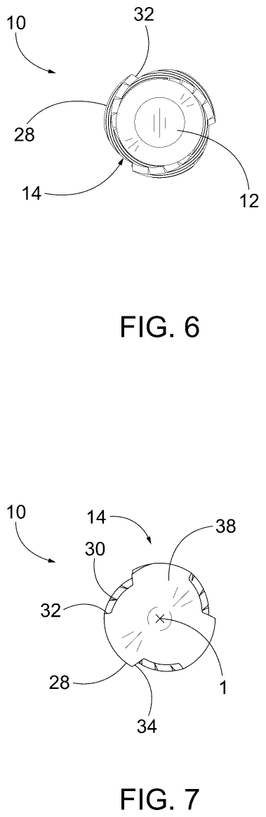

The surgical device 10 may include features to assist in cutting tissue or bone. For instance, the burr 14 includes a set of cutting flutes 24, one or more grooves 26, and cutting grooves 36. The cutting flutes 24 extend from the leading end 20 toward the trailing end 22 and spiral around the central axis 1. As shown in FIGS. 6 and 7, there are three cutting flutes 24. The burr 14 has outward surface 28, side walls 32, 34 that extend from the outward surface 28 toward an inward lower surface 30. The side walls 32, 34 may be angled relative the inward lower surface 30. Thus, the flutes taper toward each other at they extend outward as they twist around the burr 14, and the grooves 26 taper inward toward the central axis 1 as they also twist around the burr 14. The grooves 26 therefore extend into the burr body and twist along an outer surface thereof. Additional cutting grooves 36 twist around the surface of the burr and are generally transverse to the flutes 24.

In the embodiment shown in FIGS. 1-7, the leading end of burr 110 has a distal-most surface 38 that has a conical shape. The distal-most surface 38 is defined by the leading end 20 of burr 14. As shown in FIGS. 1-7, the distal most surface is conical shaped and tapers toward the central axis 1.

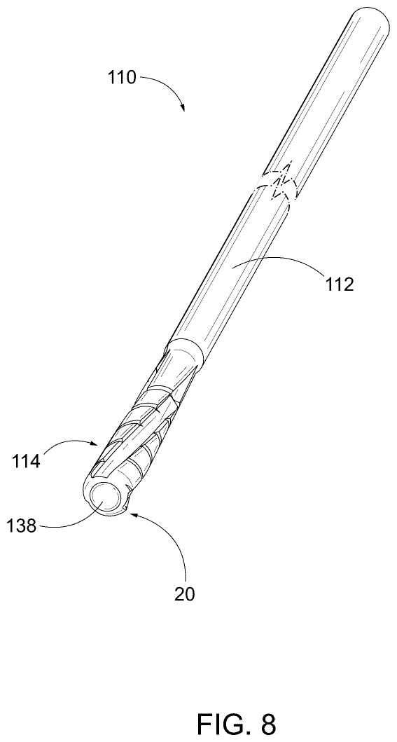

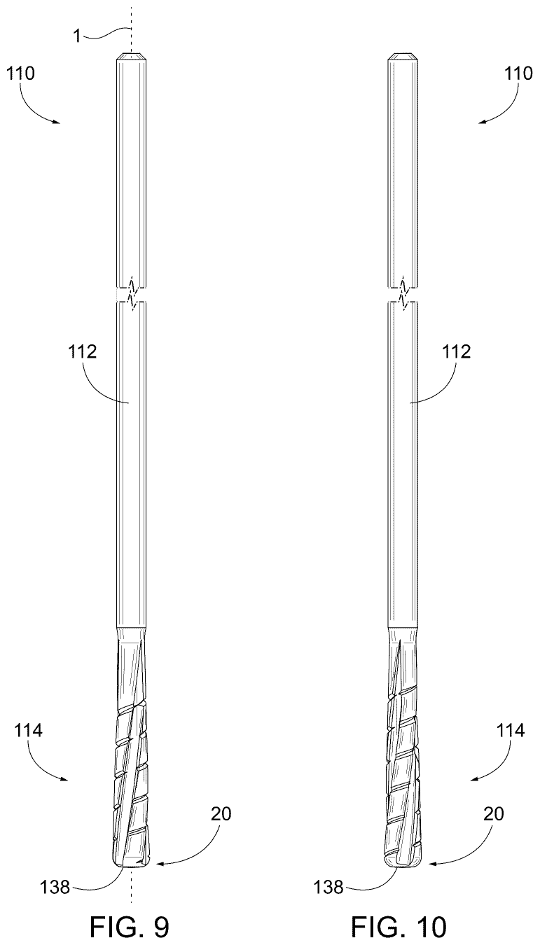

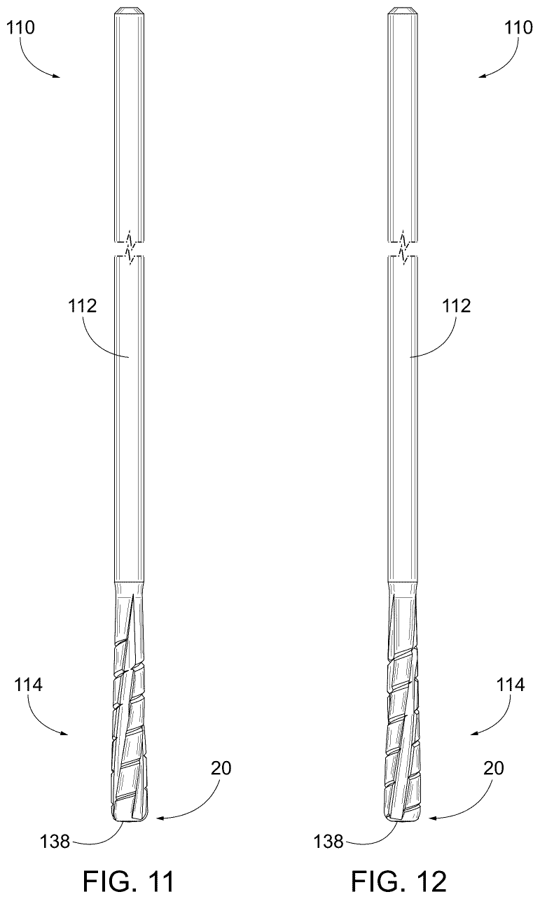

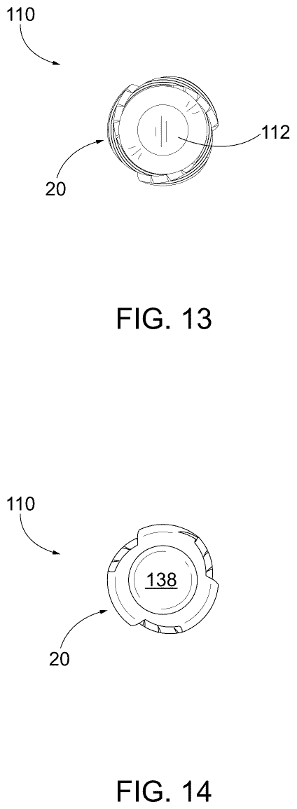

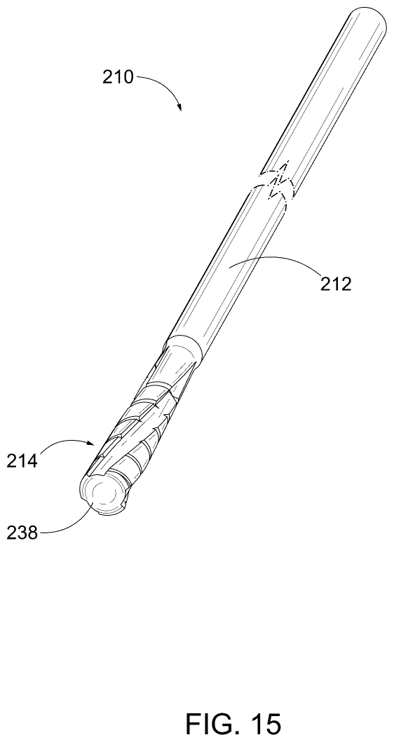

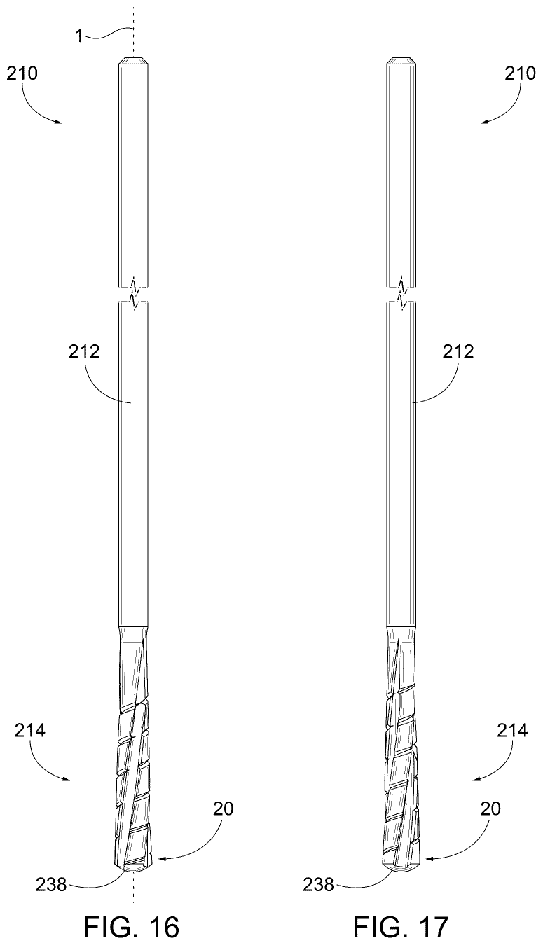

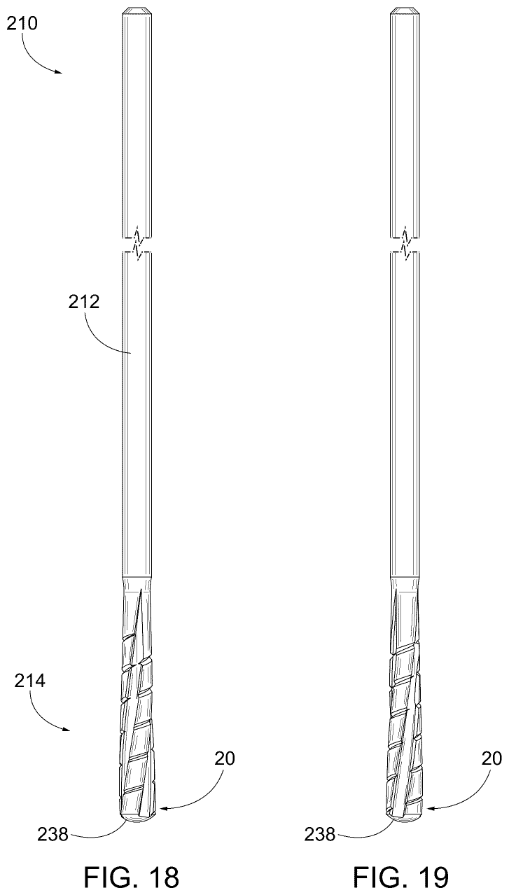

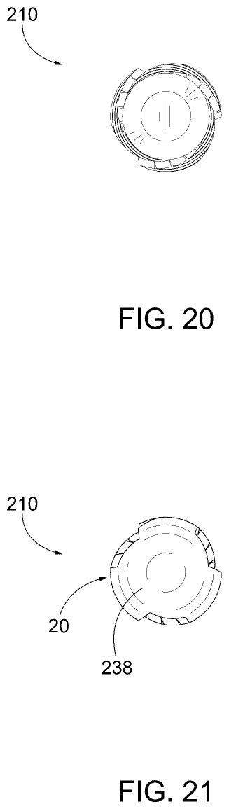

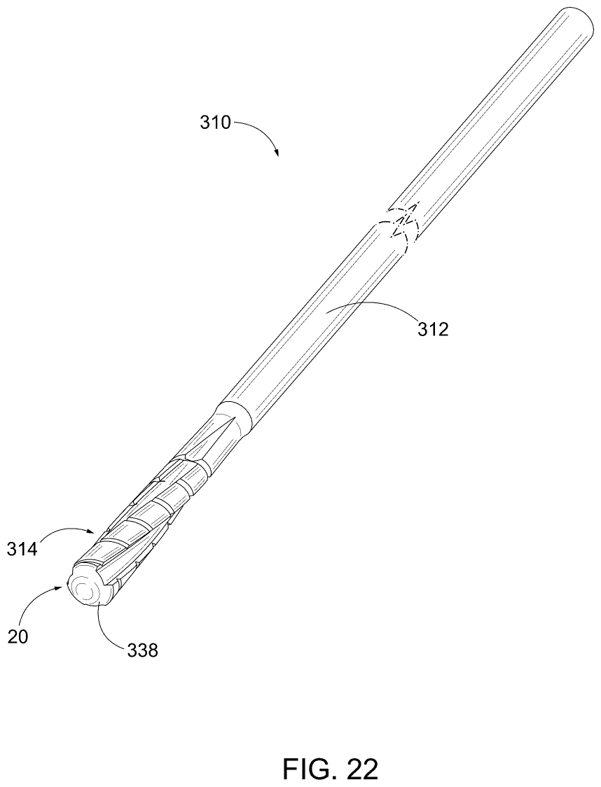

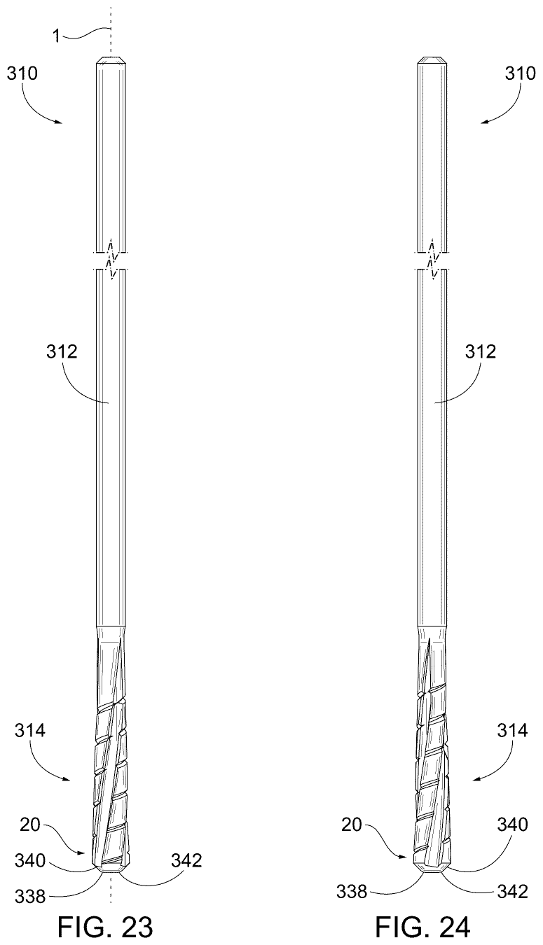

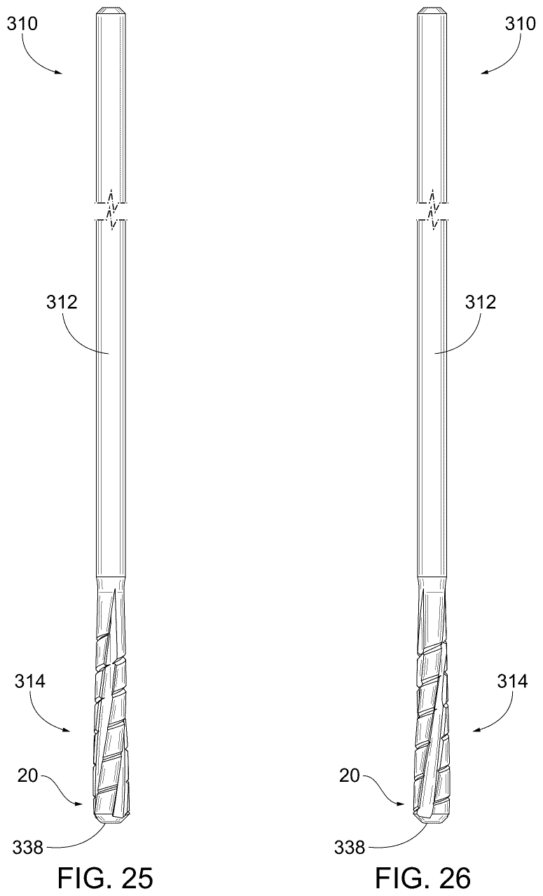

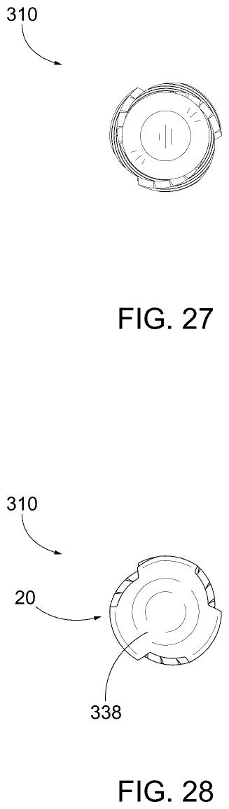

FIGS. 8-28 illustrate alternative embodiments of the burr of the surgical device. In the embodiment shown in FIGS. 8-28, the devices 110, 210, 310 are similar to the surgical device 10 described above and shown in FIG. 1-7. Features that are common between device 10 and devices 110, 210, 310 have the same reference numbers. Thus, the dimensions of the burr 14 and shaft 12, flutes 24, grooves 26, cutting grooves 36, are similar between among all devices 10, 110, 210, and 310. The embodiments shown in FIGS. 1-28 have similar burr profiles with cross-sectional dimensions that vary along the length of the burr. More specifically, the leading end of the burr has a greater-cross-sectional dimension than the remaining proximal portions of the burr. In the embodiment shown in FIGS. 8-14, the leading end 20 of burr 114 has a distal-most surface 138 that is blunt. In other words, the distal-most surface 138 is substantially perpendicular to the central axis 1. In the embodiment shown in FIGS. 15-21, the leading end 20 of burr 214 has a distal-most surface 238 has a semi-spherical shape relative to the central axis 1. In the embodiment shown in FIGS. 22-28, the leading end 20 of burr 314 has a distal-most surface 338 that has a frustoconical shape. In other words, the distal-most surface 338 has a sidewall 340 that tapers inwardly toward the central axis 1 with the planar surface 342 that intersects the side walls 338 and is perpendicular to the central axis 1.

Another embodiment includes a surgical method. The method also includes coupling a surgical device to a driver, where the surgical device has a shaft and a burr with the burr having a leading end, a trailing end, cutting flutes that extend from the leading end toward the trailing end and spiral around the central axis, and a cross-sectional dimension that his perpendicular to the central axis. Here, the leading end of the burr has a greater cross-sectional dimension that any portion of the burr head that is proximal to the leading end. The method also includes positioning the burr into the target site of an extremity bone. The method also includes powering the driver to cause the burr to resect or cut bone. The method may include distracting a joint of the target site before positioning the burr into the target site. Positioning the burr into the target site further includes distracting a joint at the target site.

The present disclosure and embodiment disclosed herein are embodied in other specific forms without departing from the spirit or essential attributes thereof and that reference should be made to the appended claims, rather than to the foregoing specification, as defining the scope of the invention.

1. A surgical device, comprising:
a shaft elongated along a central axis, the shaft having a distal end and a proximal end spaced from the distal end along the central axis in a proximal direction; and
a burr at the distal end of the shaft, the burr having a body defining a leading end, a trailing end, cutting flutes that extend from the leading end toward the trailing end and spiral around the central axis, and a cross-sectional dimension that his perpendicular to the central axis, wherein the leading end of the burr has a greater cross-sectional dimension than any portion of the burr that is proximal to the leading end.

2. The surgical device according to claim 1, further comprising a groove that extends into the body of the burr and twists along an outer surface thereof.

3. The surgical device according to claim 2, wherein the shaft has a first cross-sectional dimension, the leading end of the burr has a second cross-sectional dimension that is greater than the first cross-sectional dimension, and the trailing end of the burr has a third cross-sectional dimension that is less than the second cross-sectional dimension of the burr.

4. The surgical device according to claim 3, wherein the second cross-sectional dimension of the leading end is between 1.5 mm and 4 mm.

5. The surgical device according to claim 1, wherein the leading end of the burr has a distal-most surface, wherein the distal-most surface is blunt.

6. The surgical device according to claim 1, wherein the distal-most surface is substantially perpendicular to the central axis.

7. The surgical device according to claim 1, wherein the leading end of burr has a distal-most surface, wherein the distal-most surface defines a semi-spherical shape.

8. The surgical device according to claim 1, wherein the leading end of burr has a distal-most surface, wherein the distal-most surface defines a conical shape.

9. The surgical device according to claim 1, wherein the leading end of burr has a distal-most surface, wherein the distal-most surface defines a frustoconical shape.

10. A surgical device, comprising:
a power driver with a motor and a coupling;
a surgical burr having a) a shaft elongated along a central axis, the shaft having a distal end and a proximal end spaced from the distal end along the central axis in a proximal direction, wherein the distal end is engageable with the coupling of the power driver, and b) a burr at the distal end of the shaft, the burr having a body defining a leading end, a trailing end, cutting flutes that extend from the leading end toward the trailing end and spiral around the central axis, and a cross-sectional dimension that his perpendicular to the central axis, wherein the leading end of the burr has a greater cross-sectional dimension than any portion of the burr that is proximal to the leading end.

11. The surgical device according to claim 10, further comprising a groove that extends into the body of the burr and twists along an outer surface thereof.

12. The surgical device according to claim 11, wherein the shaft has a first cross-sectional dimension, the leading end of the burr has a second cross-sectional dimension that is greater than the first cross-sectional dimension, and the trailing end of the burr has a third cross-sectional dimension that is less than the second cross-sectional dimension of the burr.

13. The surgical device according to claim 12, wherein a cross-sectional dimension of the leading end is between 1.5 mm and 4 mm.

14. The surgical device according to claim 10, wherein the leading end of the burr has a distal-most surface, wherein the distal-most surface is blunt.

15. The surgical device according to claim 14, wherein the distal-most surface is substantially perpendicular to the central axis.

16. The surgical device according to claim 10, wherein the leading end of burr has a distal-most surface, wherein the distal-most surface defines a semi-spherical shape.

17. The surgical device according to claim 16, wherein the leading end of burr has a distal-most surface, wherein the distal-most surface defines a conical shape.

18. The surgical device according to claim 17, wherein the leading end of burr has a distal-most surface, wherein the distal-most surface defines a frustoconical shape.

19. A method, comprising:
coupling a surgical burr to a driver, wherein the surgical burr, the surgical burr having a leading end, a trailing end, cutting flutes that extend from the leading end toward the trailing end and spiral around a central axis, and a cross-sectional dimension that his perpendicular to the central axis, wherein the leading end of the surgical burr has a greater cross-sectional dimension that any portion of the surgical burr that is proximal to the leading end;
positioning the burr into a target site of an extremity bone; and
powering the driver to cause the burr to resect or cut the extremity bone.

20. The method of claim 19, further comprising distracting a joint of the target site before positioning the surgical burr into the target site.

21. The method of claim 19, further comprising a groove that extends into the body of the burr and twists along an outer surface thereof.

22. The method of claim 19, wherein the shaft has a first cross-sectional dimension, the leading end of the burr has a second cross-sectional dimension that is greater than the first cross-sectional dimension, and the trailing end of the burr has a third cross-sectional dimension that is less than the second cross-sectional dimension of the burr.
